(19) United States
(12) Patent Application Publication
(10) Pub. No.: US 2025/0302488 A1
(43) Pub. Date: Oct. 2, 2025

(54) KINEMATICALLY ALIGNED ORTHOPAEDIC SURGICAL INSTRUMENT AND METHOD OF USING THE SAME IN AN ORTHOPAEDIC KNEE PROCEDURE

(71) Applicant: DePuy Ireland Unlimited Company, Ringaskiddy (IE)

(72) Inventor: Thomas E. Wogoman, Warsaw, IN (US)

(21) Appl. No.: 19/070,936

(22) Filed: Mar. 5, 2025

Related U.S. Application Data
(60) Provisional application No. 63/570,978, filed on Mar. 28, 2024.

(51) Int. Cl. A61B 17/15; A61B 17/56; A61F 2/46

(52) U.S. Cl. CPC A61B 17/155; A61F 2/461; A61B 2017/564; A61F 2002/4687

(57) ABSTRACT

An orthopaedic surgical instrument includes a 4-in-1 cutting block and a removable alignment guide. The alignment guide is configured to contact the posterior femoral condyles of a patient's femur so as to kinematically align the 4-in-1 cutting block. A number of shims are selectively installed on the alignment guide to account for any cartilage loss present on the patient's femur. A method of surgically preparing a patient's femur utilizing kinematic alignment is also disclosed.

This application claims priority under 35 U.S.C. § 119 to U.S. Provisional Patent Application Ser. No. 63/570,978 which was filed on Mar. 28, 2024, and is hereby incorporated by reference in its entirety.

### TECHNICAL FIELD

The present disclosure relates generally to orthopaedic surgical instruments and, more particularly, to surgical instruments used to resect a patient's bone.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

Kinematic alignment is a method of total knee arthroplasty (TKA) that involves resecting the femur and tibia as a function of their native, pre-disease state. Traditional TKA involves determining the resection planes based on a pre-determined angle as a function of mechanical alignment or by using a balanced approach that sets the rotation based on ligament tension.

### SUMMARY

According to one aspect of the disclosure, an orthopaedic surgical instrument for use in the surgical preparation of a patient's femur during performance of an orthopaedic knee procedure includes a 4-in-1 femoral cutting block having defined therein a pair of chamfer cutting slots, an anterior cutting slot spaced apart anteriorly from the pair of chamfer cutting slots, and a posterior cut surface spaced apart posteriorly from the pair of chamfer cutting slots. A medial paddle is spaced apart posteriorly from the posterior cut surface. The medial paddle has an alignment surface that extends parallel to the posterior cut surface of the 4-in-1 femoral cutting block in the superior/inferior direction and is configured to be positioned in contact with a medial posterior condyle of the patient's femur. A lateral paddle is spaced apart posteriorly from the posterior cut surface. The lateral paddle has an alignment surface that extends parallel to the posterior cut surface of the 4-in-1 femoral cutting block in the superior/inferior direction and is configured to be positioned in contact with a lateral posterior condyle of the patient's femur.

The orthopaedic surgical instrument also includes a plurality of shims. Each of the plurality of shims has a thickness that is different from the thickness of at least some of the other of the plurality of shims. In one embodiment, each of the plurality of shims includes a locking mechanism that mates with a locking mechanism of the medial paddle and the lateral paddle such that each of the plurality of shims is configured to be separately coupled to both the medial paddle and the lateral paddle. In another embodiment, each of the plurality of shims includes a locking mechanism that mates with a locking mechanism of one or both of the medial paddle and the lateral paddle such that each of the plurality of shims is configured to be separately coupled to one or both of the medial paddle and the lateral paddle.

The orthopaedic surgical instrument also includes a posterior saw capture surface. The posterior saw capture surface is spaced apart posteriorly from the posterior cut surface and anteriorly from both the medial paddle and the lateral paddle.

The orthopaedic surgical instrument may also include an alignment guide that is selectively securable to, and removable from, the 4-in-1 femoral cutting block. In such an embodiment, each of the medial paddle, the lateral paddle, and the saw capture surface are formed in the alignment guide.

The alignment guide may include a connector configured to secure the alignment guide to the 4-in-1 femoral cutting block.

In an embodiment, the medial paddle has a longitudinal axis extending in the superior/inferior direction, and the lateral paddle has a longitudinal axis that also extends in the superior/inferior direction so as to be parallel to the longitudinal axis of the medial paddle.

According to another aspect, an orthopaedic surgical instrument for use in the surgical preparation of a patient's femur during performance of an orthopaedic knee procedure includes a 4-in-1 femoral cutting block and an alignment guide. The 4-in-1 femoral cutting block has defined therein a pair of chamfer cutting slots, an anterior cutting slot spaced apart anteriorly from the pair of chamfer cutting slots, and a posterior cut surface spaced apart posteriorly from the pair of chamfer cutting slots. The alignment guide is removably secured to a posterior end of the 4-in-1 femoral cutting block. The alignment guide includes a medial paddle that is spaced apart posteriorly from the posterior cut surface. The medial paddle also has an alignment surface that extends parallel to the posterior cut surface of the 4-in-1 femoral cutting block in the superior/inferior direction and is configured to be positioned in contact with a medial posterior condyle of the patient's femur. The alignment guide also includes a lateral paddle spaced that is apart posteriorly from the posterior cut surface. The lateral paddle has an alignment surface that extends parallel to the posterior cut surface of the 4-in-1 femoral cutting block in the superior/inferior direction and is configured to be positioned in contact with a lateral posterior condyle of the patient's femur.

The orthopaedic surgical instrument also includes a plurality of shims. Each of the plurality of shims has a thickness that is different from the thickness of at least some of the other of the plurality of shims. In one embodiment, each of the plurality of shims includes a locking mechanism that mates with a locking mechanism of the medial paddle and the lateral paddle such that each of the plurality of shims is configured to be separately coupled to both the medial paddle and the lateral paddle. In another embodiment, each of the plurality of shims includes a locking mechanism that mates with a locking mechanism of one or both of the medial paddle and the lateral paddle such that each of the plurality of shims is configured to be separately coupled to one or both of the medial paddle and the lateral paddle.

The alignment guide may also include a posterior saw capture surface. The posterior saw capture surface is spaced apart posteriorly from the posterior cut surface of the 4-in-1 femoral cutting block and anteriorly from both the medial paddle and the lateral paddle.

The alignment guide may include a connector configured to secure the alignment guide to the 4-in-1 femoral cutting block.

In an embodiment, the medial paddle has a longitudinal axis extending in the superior/inferior direction, and the lateral paddle has a longitudinal axis that also extends in the superior/inferior direction so as to be parallel to the longitudinal axis of the medial paddle.

In yet another aspect, a method of surgically preparing a patient's femur during performance of an orthopaedic knee procedure includes resecting a distal end of the patient's femur so as to create a resected planar surface, and determining an amount of cartilage loss on a posterior femoral condyle of the patient's femur. A shim having a thickness that corresponds to the determined amount of cartilage loss on the posterior femoral condyle of the patient's femur is then selected from a plurality of shims of differing thicknesses. The selected shim is then installed onto a paddle positioned on a posterior end of a 4-in-1 femoral cutting block. The 4-in-1 femoral cutting block is then positioned on the resected distal end of the patient's femur such that (i) a backside surface of the 4-in-1 femoral cutting block contacts the resected planar surface, and (ii) the shim installed on the paddle contacts the posterior femoral condyle of the patient's femur.

The 4-in-1 femoral cutting block may be pinned to the patient's femur subsequent to positioning thereof on the resected distal end of the patient's femur. Thereafter, further resections may be performed on the patient's femur by use of the pinned 4-in-1 femoral cutting block. Such further resections may include an anterior resection, a distal resection, and a pair of chamfer resections.

In some cases, the amount of cartilage loss is determined on the medial posterior femoral condyle of the patient's femur. A corresponding shim is then selected and installed on a medial paddle positioned on a posterior end of a 4-in-1 femoral cutting block and thereafter positioned in contact with the medial posterior femoral condyle of the patient's femur.

In some cases, the amount of cartilage loss is determined on the lateral posterior femoral condyle of the patient's femur. A corresponding shim is then selected and installed on a lateral paddle positioned on a posterior end of a 4-in-1 femoral cutting block and thereafter positioned in contact with the lateral posterior femoral condyle of the patient's femur.

In some cases, the amount of cartilage loss is determined on both the medial and lateral posterior femoral condyles of the patient's femur. A corresponding shim is then selected and installed on both the medial and lateral paddles positioned on a posterior end of a 4-in-1 femoral cutting block and thereafter positioned in contact with both the medial and lateral posterior femoral condyle of the patient's femur.

In an embodiment, a depth probe is advanced into cartilage of the posterior femoral condyle of the patient's femur to determine the amount of cartilage loss.

### BRIEF DESCRIPTION OF THE DRAWINGS

The detailed description particularly refers to the following figures, in which:

FIG. 1 is a perspective view of an alignment guide of an orthopaedic surgical instrument for use in the surgical preparation of a patient's femur during performance of a kinematic alignment orthopaedic knee procedure;

FIGS. 2 and 3 are a perspective views showing the alignment guide of FIG. 1 secured to a 4-in-1 cutting block;

FIG. 4 is a side view showing the alignment guide of FIG. 1 secured to a 4-in-1 cutting block;

FIG. 5 is a perspective view showing a depth probe inserted into a posterior condyle of the patient's femur subsequent to performance of a distal resection of the patient's femur;

FIG. 6 is a perspective view showing the shims being assembled to the alignment guide; and

FIGS. 7 and 8 show the alignment guide secured to the 4-in-1 cutting block and being used to align the cutting block to the resected distal end of the patient's femur.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

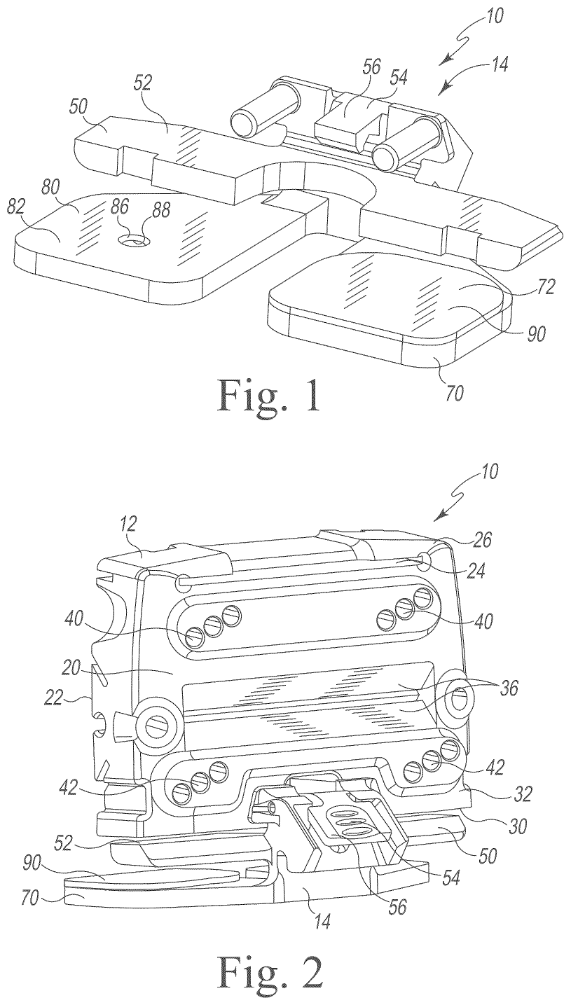

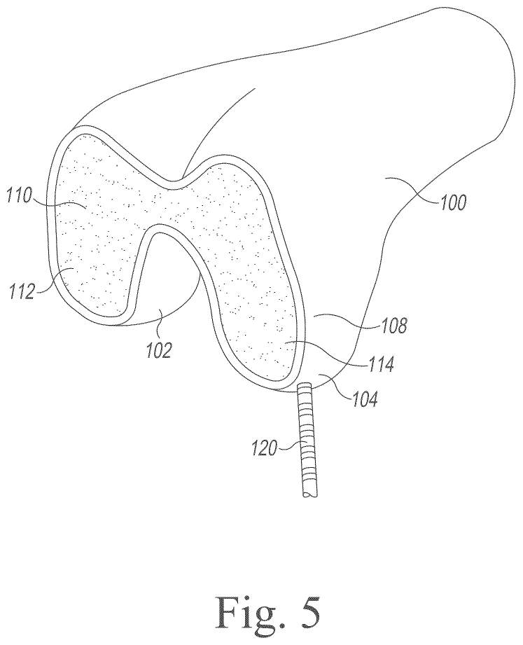

Referring to FIGS. 1-4, an orthopaedic surgical instrument 10 for use in the surgical preparation of a patient's femur during performance of a kinematic alignment orthopaedic knee procedure is shown. In such a kinematic alignment orthopaedic knee procedure, the location of the resections made on the patient's femur are selected as a function of the native, pre-disease state of the patient's knee. In practice, this is done by performing a series of resections on the bone generally parallel to the articular surface while compensating for cartilage wear. The prosthetic femoral implant component implanted on such resections will thus be positioned as a function of the patient's knee anatomy prior to the onset of arthritis or occurrence of trauma.

The orthopaedic instrument 10 includes a 4-in-1 femoral cutting block 12 and a removable alignment guide 14. The alignment guide 14 is used to kinematically align the 4-in-1 cutting block 12 so that the block 12 may be used in the surgical preparation of the patient's distal femur during a knee replacement procedure. As will be discussed below in greater detail, the 4-in-1 cutting block 12 is used to perform four cuts on the patient's distal femur with the same block—an anterior cut, a posterior cut, and two chamfer cuts—to prepare the distal femur to receive a prosthetic femoral implant component. However, it should be appreciated that although the femoral cutting block of the orthopaedic surgical instrument 10 is herein described as a 4-in-1 cutting block, and has significant advantages thereby in the design of the instrument, the femoral cutting block may be embodied as other types of cutting blocks and still enjoy certain of such advantages. For example, the orthopaedic surgical instrument 10 may be embodied with a 2-in-1 cutting block or a 5-in-1 cutting block. Yet further, in lieu of a cutting block, the orthopaedic surgical instrument 10 may be embodied with a pin guide that is kinematically aligned on the patient's femur by use of the alignment guide 14 and thereafter used to install a pair of guide pins on the patient's femur.

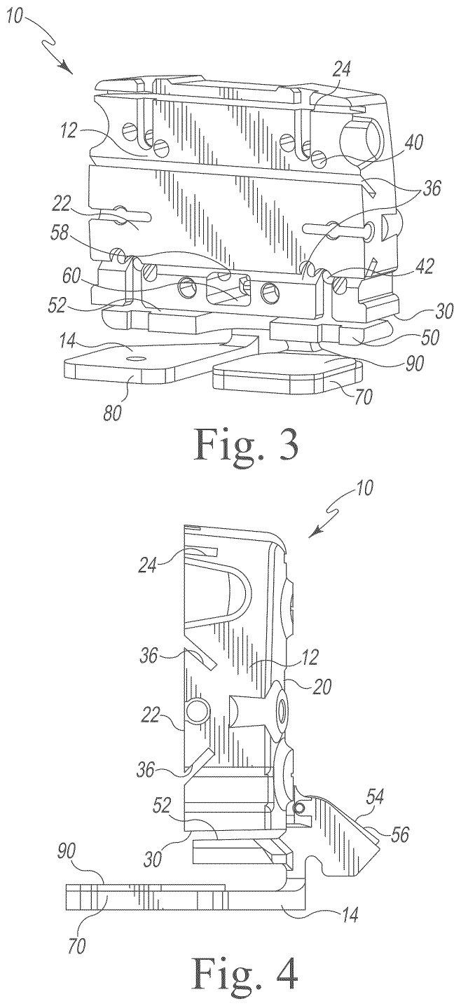

As can be seen in FIG. 2-4, the 4-in-1 cutting block 12 includes an outer surface 20 and a bone-facing backside surface 22 positioned opposite the outer surface 20. The 4-in-1 cutting block 12 has an anterior cutting slot 24 formed near its anterior end 26. The anterior cutting slot 24 is an elongated slot extending in the medial/lateral direction. The anterior cutting slot 24 extends through the entire thickness of the 4-in-1 cutting block 12—that is, the anterior cutting slot 24 extends from the cutting block's outer surface 20 to its bone-facing backside surface 22 thereby being open to both surfaces. The anterior cutting slot 24 is embodied as a captured cutting guide (i.e., it is closed on all sides so as to capture a saw blade therein), although the cutting block 12 and the cutting slot 24 may alternatively be embodied as a non-captured cutting guide. The anterior cutting slot 24 is sized and shaped to receive the blade of a surgical saw or other cutting instrument and orient the blade to resect the anterior surface of the patient's femur during an orthopaedic surgical procedure.

The 4-in-1 cutting block 12 also has a posterior cutting surface 30 formed near its posterior end 32. The posterior cutting surface 30 is an elongated surface extending in the medial/lateral direction. The posterior cutting surface 30 extends the entire thickness of the 4-in-1 cutting block 12—that is, it extends from the cutting block's outer surface 20 to its bone-facing backside surface 22. The posterior cutting surface 30 is sized and shaped to support and guide the blade of a surgical saw or other cutting instrument and orient the blade to resect the posterior surface of the patient's femur during an orthopaedic surgical procedure. Moreover, as will be discussed below in more detail, the alignment guide 14 includes a saw capture cutting guide 50 having a posterior cutting surface 52. When the alignment guide 14 is secured to the 4-in-1 cutting block 12, the alignment guide's posterior cutting surface 52 cooperates with the block's posterior cutting surface 30 to form a captured cutting guide.

The 4-in-1 cutting block 12 also has a pair of chamfer cutting slots 36 formed near its middle. Specifically, the chamfer cutting slots 36 are located posteriorly of the anterior cutting slot 24 and anteriorly of the posterior cutting surface 30. Each of the chamfer cutting slots 36 is an elongated slot extending in the medial/lateral direction. The chamfer cutting slots 36 extend through the entire thickness of the 4-in-1 cutting block 12—that is, each of the slots 36 extends from the cutting block's outer surface 20 to its bone-facing backside surface 22 and, as a result, opens to both surfaces. The chamfer cutting slots 36 are sized and shaped to receive the blade of a surgical saw or other cutting instrument and orient the blade to resect the anterior and posterior surfaces of the patient's femur during an orthopaedic surgical procedure. In particular, one of the chamfer cutting slots 36 is configured to guide a saw blade during performance of the anterior chamfer cut, with the other chamfer cutting slot 36 being configured to guide the saw blade during performance of the posterior chamfer cut.

The 4-in-1 cutting block 12 has a plurality of guide holes 40 defined therein that are sized to receive a pair of fixation or guide pins (not shown). The guide holes 40 are positioned between the anterior cutting slot 24 and the chamfer cutting slots 36 and extend between the outer surface 20 and the bone-facing backside surface 22 of the cutting block 12. The holes 40 are arranged in a staggered pattern to permit the surgeon to change the position of the cutting block 12 on the patient's femur without having to remove the fixation pins.

The 4-in-1 cutting block 12 also includes another plurality of guide holes 42 positioned between the chamfer cutting slots 36 and the posterior cutting surface 30. Each guide hole 42 is sized to receive one of the fixation pins in a similar manner to the guide holes and thereby extends between the outer surface 20 and the bone-facing backside surface 22 of the cutting block 12. Like the guide holes 40, the guide holes 42 are arranged in a staggered pattern to permit the surgeon to change the position of the cutting block 12 on the patient's femur without having to remove the fixation pins.

As alluded to above and as can be seen in FIGS. 1-4, the alignment guide 14 includes a saw capture cutting guide 50 having a posterior cutting surface 52. When the alignment guide 14 is secured to the 4-in-1 cutting block 12, the alignment guide's posterior cutting surface 52 cooperates with the block's posterior cutting surface 30 to form a captured cutting guide. The alignment guide 14 also includes a connector 54 that is configured to secure the alignment guide 14 to the 4-in-1 cutting block 12 so that the guide 14 may be selectively attached to, and removed from, the block 12. In an illustrative embodiment, the connector 54 is embodied as a spring-loaded pushbutton clip 56 that is received into an opening 58 formed in the 4-in-1 cutting block 12 and clips to a retaining surface 60 defined in the block 12 (see FIG. 3), along with a pair of pins on either side of the clip 56. A surgeon or other user may press the pushbutton, advance the clip 56 into the opening 58, and thereafter release the pushbutton to secure the alignment guide 14 to the 4-in-1 cutting block 12. To remove the alignment guide 14 from the 4-in-1 cutting block 12, the surgeon or other user presses the pushbutton and pulls the clip 56 from the opening 58 and thus free of the 4-in-1 cutting block 12. It should be appreciated that although the alignment guide 14 is herein described as a separate instrument that is removable from the 4-in-1 cutting block 12, the features of the alignment guide 14 may be incorporated into the design of the 4-in-1 cutting block 12 to form a common integrated instrument.

It should be appreciated that some designs of the 4-in-1 cutting block 12 may include a captured posterior cutting guide (and hence not utilize a removable saw capture cutting guide 50 to form such a captured posterior cutting guide). In such a case, the alignment guide 14 may be embodied with a connector in the form of a blade that is inserted into, and frictionally retained within, the integral captured posterior cutting guide.

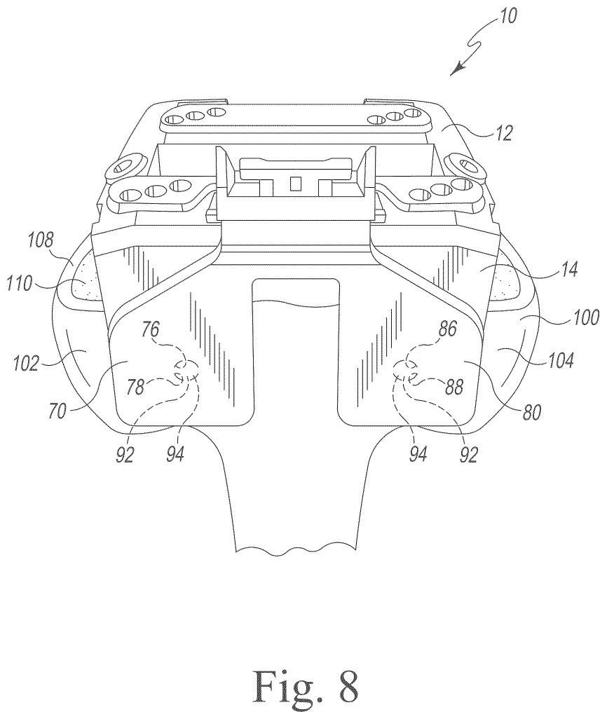

The alignment guide 14 also includes a medial paddle 70 and a lateral paddle 80. When the alignment guide 14 is secured to the 4-in-1 cutting block 12, the paddles 70, 80 are positioned below the posterior end 32 of the block 12 and thus spaced apart posteriorly from the posterior cut surface 30. As can be seen in FIGS. 3, 4, 7, and 8. the medial paddle 70 has an alignment surface 72 that extends parallel to the posterior cut surface 30 of the 4-in-1 femoral cutting block 12 in the superior/inferior direction. As shown in FIGS. 7 and 8, the alignment surface 72 of the medial paddle 70 is configured to be positioned in contact with a medial posterior condyle 102 of the patient's femur 100. As will be described below in greater detail, in some instances, the alignment surface 72 may also include a shim when positioned in contact with the medial posterior condyle 102 of the patient's femur 100. Similarly, the lateral paddle 80 has an alignment surface 82 that extends parallel to the posterior cut surface 30 of the 4-in-1 femoral cutting block 12 in the superior/inferior direction. As shown in FIGS. 7 and 8, the alignment surface 82 of the lateral paddle 80 is configured to be positioned in contact with a lateral posterior condyle 104 of the patient's femur 100. Similarly to the medial paddle 70, in some instances, the alignment surface 82 of the lateral paddle 80 may also include a shim when positioned in contact with the lateral posterior condyle 104 of the patient's femur 100.

As can be seen in FIGS. 1-3, in the illustrative embodiment of the orthopaedic surgical instrument 10 described herein, the alignment surfaces 72, 82 of the paddles 70, 80 extend parallel to the alignment guide's posterior cutting surface 52 and the block's posterior cut surface 30 in the medial/lateral direction. However, the alignment guide 14 may be configured such that the alignment surfaces 72, 82 of the paddles 70, 80 extend at a slight angle (e.g., 1-3 degrees) relative to the posterior cut surfaces 30, 52 in the medial/lateral direction. Depending on the direction of inclination, such a slight angle may be utilized to remove a small amount of natural valgus or varus. In particular, some patients have a knee anatomy with abnormal amounts of natural valgus or varus. Instead of restoring these abnormal amounts of natural valgus or varus, use of angled alignment surfaces 72, 82 of the paddles 70, 80 allows the surgeon to remove such an abnormality while still restoring the remainder of the patient's knee anatomy to its original, pre-diseased state.

As can be seen in FIGS. 3, 6, and 8, the medial paddle 70 and the lateral paddle 80 extend parallel to one another in the superior/inferior direction. Specifically, the longitudinal axis 74 of the medial paddle 70 extends in the superior/inferior direction and is parallel to the longitudinal axis 84 of the lateral paddle 80 which also extends in the superior/inferior direction. Such an arrangement allows the alignment surfaces 72, 82 of the paddles 70, 80 to be positioned in the desired orientation for contact with the posterior condyles 102, 104 of the patient's femur 100, respectively.

As alluded to above, the orthopaedic surgical instrument 10 also includes a number of shims 90. This shims 90 are configured with varying different sizes (i.e., different thicknesses) so that the surgeon can intraoperatively select a best-fit option to pair with the paddles 70, 80 of the alignment guide 14. For example, the shims 90 of the orthopaedic surgical instrument 10 may be provided in size 1 (e.g., having a 1 mm thickness), size 2 (e.g., having a 2 mm thickness), and size 3 (e.g., having a 3 mm thickness). Shims 90 of additional sizes may also be provided to fit the needs of a given orthopaedic surgical instrument 10.

Each of the shims 90 includes a locking mechanism 92 that mates with a locking mechanism 76, 86 of the alignment guide's paddles 70, 80, respectively. In the illustrative embodiment described herein, the locking mechanism 92 of the shims 90 is embodied as a post 94 and the locking mechanisms 76, 86 of the alignment guide's paddles 70, 80 are embodied as a pair of elongated bores 78, 88, respectively, extending through the paddles 70, 80. The post 94 is sized to be press fit into the elongated bores 78, 88 to couple the shim 90 to the paddles 70, 80. In the illustrative embodiment described herein, the shims 90 are “universal” (i.e., not side-specific). In other words, each of the shims 90 may be coupled to either the medial paddle 70 or the lateral paddle 80. However, in some embodiments, the shims 90 may be side-specific and, as a result, specifically-shaped to be coupled to one of the paddles 70, 80, but not both.

As will be described below in greater detail, during performance of a surgical procedure to prepare the distal end 108 of the patient's femur 100, the surgeon determines or otherwise estimates an amount of cartilage loss on one or both of the medial posterior condyle 102 and the lateral posterior condyle 104 of the patient's femur 100. The surgeon may then utilize a shim 90 that has a size (i.e., thickness) that corresponds to the determined amount of cartilage loss on the medial posterior condyle 102 and/or the lateral posterior condyle 104 of the patient's femur 100. In particular, the surgeon selects a shim 90 having a size (i.e., thickness) that corresponds with the amount of cartilage loss from the differently sized shims 90 and installs the selected shim 90 on the corresponding paddle 70, 80.

In operation, the surgeon may utilize the orthopaedic surgical instrument 10 during performance of an orthopaedic knee procedure to prepare the distal end 108 of the patient's femur 100 to receive a prosthetic femoral component. To do so, the surgeon may utilize the alignment guide 14 to secure the 4-in-1 cutting block 12 utilizing kinematic alignment to the patient's femur 100 and thereafter use the cutting guides of the cutting block 12 to guide a bone saw blade in making a series of four resections of the distal end 108 of the patient's femur 100.

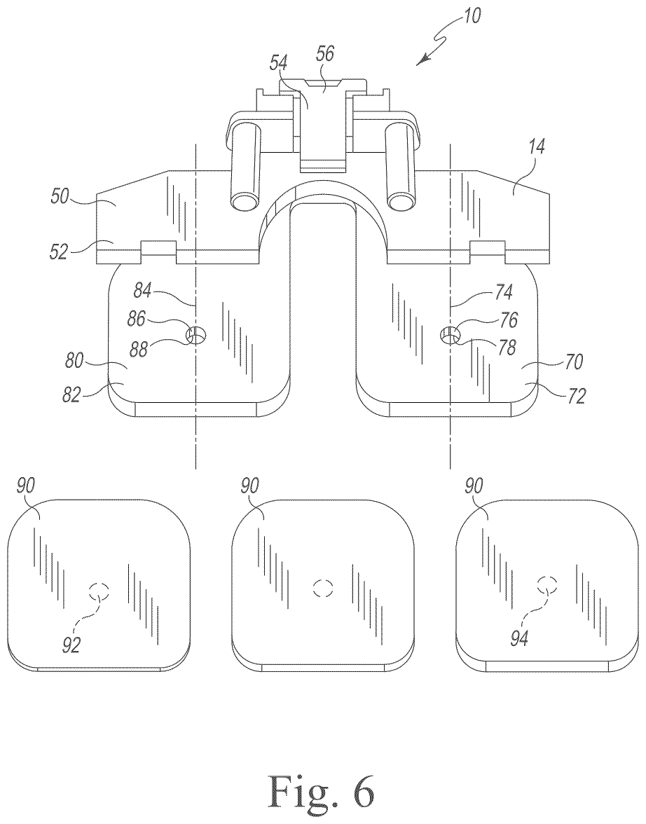

As shown in FIG. 5, during such an orthopaedic surgical procedure, the surgeon may first resect the distal end 108 of the patient's femur 100 to create a surgically-prepared planar distal surface 110 that includes a planar medial condylar surface 112 and a planar lateral condylar surface 114. To do so, the surgeon may use a cutting guide assembly such as the Attune® Distal Femoral Jig and the Attune® Distal Femoral Cutting Block, both of which are commercially available from DePuy Synthes of Warsaw, Indiana. The surgeon may then determine an amount of cartilage loss on each of the posterior femoral condyles 102, 104 of the patient's femur 100. To do so, the surgeon determines the amount of cartilage loss on an affected area of the medial posterior femoral condyle 102 (i.e., an area of the condyle 102 exhibiting cartilage loss) by inserting a graduated depth probe 120 or similar instrument into the affected area of the posterior femoral condyle 102 and then also inserting the graduated depth probe 120 into an unaffected area of the medial posterior femoral condyle 102 (i.e., an area of the condyle 102 that does not exhibit cartilage loss). The difference between the two depth measurements reflects the amount of cartilage loss on the medial posterior femoral condyle 102. The surgeon then repeats the process on the lateral posterior femoral condyle 104. Specifically, the surgeon inserts the graduated depth probe 120 into both the affected area of the lateral posterior femoral condyle 104 and an unaffected area of the lateral posterior femoral condyle 104 with the difference between the two depth measurements reflecting the amount of cartilage loss on the lateral posterior femoral condyle 104.

The surgeon then determines the size of the prosthetic femoral component to be implanted on the patient's femur 100. To do so, the surgeon may use a sizing instrument such as the Attune® Measured Sizer instrument which is commercially available from DePuy Synthes. Alternatively, the surgeon may determine the appropriate size of the implant from preoperative planning or other intraoperative techniques. In any such case, once the surgeon determines the size of the prosthetic femoral component to be implanted, the surgeon selects the corresponding size of the 4-in-1 cutting block 12 and couples the alignment guide 14 thereto. To do so, the surgeon advances the spring-loaded pushbutton clip 56 into the opening 58 formed in the selected 4-in-1 cutting block 12 so as to clip the guide 14 to the cutting block 12.

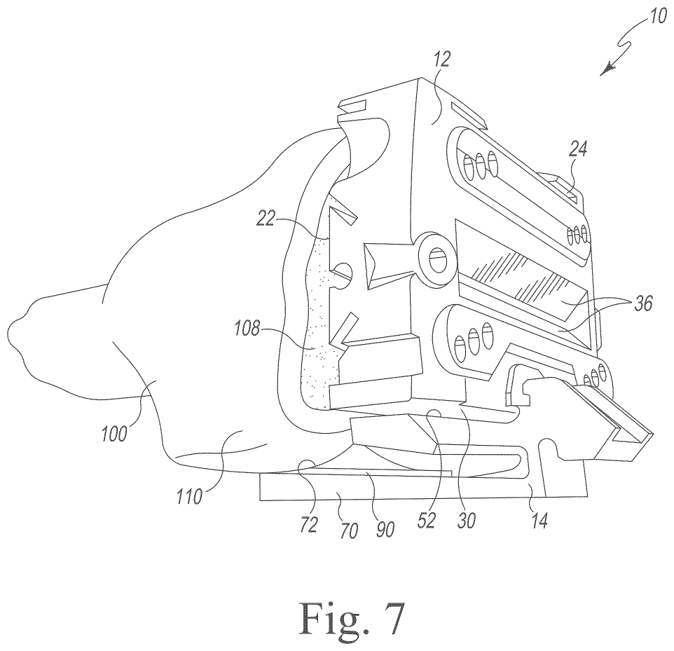

Once the surgeon has coupled the alignment guide 14 to the 4-in-1 cutting block 12, the surgeon then installs any necessary shims 90 on the alignment guide 14. Specifically, the surgeon selects an appropriately sized shim 90 for each of the paddles 70, 80 based on the determined amount of cartilage loss, if any, on the posterior femoral condyles 102, 104 (as was previously determined by use of the depth probe 120). In particular, as shown in FIG. 6, the surgeon selects a shim 90 having a size (thickness) which corresponds to the amount of cartilage loss on each of the posterior femoral condyles 102, 104 and installs it on the respective paddle 70, 80. For example, if the surgeon determines that the medial posterior femoral condyle 102 of the patient's femur has 2 mm of cartilage loss, the surgeon selects a size 2 (2 mm thick) shim 90 and installs it on the medial paddle 70 of the alignment guide 14. Similarly, for example, if the surgeon determines that the lateral posterior femoral condyle 104 of the patient's femur has 1 mm of cartilage loss, the surgeon selects a size 1 (1 mm thick) shim 90 and installs it on the lateral paddle 80 of the alignment guide 14. It should be appreciated that if the surgeon determines that either (or both) of the posterior femoral condyles 102, 104 does not exhibit any cartilage loss, a shim 90 is not installed on the respective paddle(s) 70, 80. For example, if the surgeon determines that the medial posterior femoral condyle 102 of the patient's femur has 2 mm of cartilage loss, but the lateral posterior femoral condyle 104 does not exhibit any cartilage loss, the surgeon selects a size 2 (2 mm thick) shim 90 and installs it on the medial paddle 70 of the alignment guide 14, but does not install any shims 90 on the lateral paddle 80.

Thereafter, as shown in FIGS. 7 and 8, the surgeon positions the assembled orthopaedic surgical instrument 10 on the resected distal end 108 of the patient's femur 100. In particular, the surgeon positions the instrument 10 such that the bone-facing surface 22 of the 4-in-1 cutting block 12 is positioned in contact with the resected distal end 108 of the patient's femur 100, the alignment surface 72 of the medial paddle 70 (including any shims 90 included therewith) is positioned in contact with the medial posterior condyle 102, and the alignment surface 82 of the lateral paddle 80 (including any shims 90 included therewith) is positioned in contact with the lateral posterior condyle 104. Doing so positions the 4-in-1 cutting block 12 in a position based off the original cartilage profile of the patient's femur 100 since the size (i.e., thickness) of the installed shims 90 account for the patient's cartilage loss. Once so positioned, the surgeon may use a stylus (not shown), such as the Attune® Femoral Stylus commercially available from DePuy Synthes, to confirm the anterior resection plane.

Once the surgeon is satisfied with the kinematic alignment of the 4-in-1 cutting block 12, the surgeon may pin the block 12 to the resected distal end 108 of the patient's femur 100 by installing a pair of fixation pins (not shown) through a corresponding pair of the guide holes 40. If the surgeon desires to relocate the 4-in-1 cutting block 12, the surgeon may utilize another combination of guide holes 40 to change the position of the cutting block 12 on the patient's femur 100. If additional fixation is necessary, the surgeon may insert additional fixation pins installed through the guide holes 42 of the 4-in-1 cutting block 12.

Once installed in such a manner, the surgeon may use the 4-in-1 cutting block 12 to make a number of resections of the distal end 108 of the patient's femur 100. For example, the surgeon may advance a cutting tool, such as a surgical cutting saw (not shown) through the anterior cutting slot 24 to engage the patient's femur 100 and operate the surgical saw to surgically prepare an anterior surface of the patient's femur 100 to receive the prosthetic femoral component. The surgeon may similarly use the captured cutting guide created by the alignment guide's posterior cutting surface 52 and the block's posterior cutting surface 30 to resect the posterior condyles 102, 104 of the patient's femur 100 thereby surgically preparing the posterior surfaces of the patient's femur 100 to receive the prosthetic femoral component. The surgeon may also use the chamfer cutting slots 36 to make a pair of chamfer cuts on the patient's femur 100.

Once the anterior cut, posterior cut, and both chamfer cuts have been made, the surgeon removes the orthopaedic surgical instrument 10 from the patient's femur and installs a prosthetic femoral implant component onto the resected surfaces. Since the locations of the resections made on the patient's femur were selected while compensating for cartilage wear-and thus reflect the native, pre-disease state of the patient's knee-the prosthetic femoral implant component implanted on such resections will also be positioned as a function of the patient's knee anatomy prior to the onset of arthritis or occurrence of trauma.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument for use in the surgical preparation of a patient's femur during performance of an orthopaedic knee procedure, the orthopaedic surgical instrument comprising:
a 4-in-1 femoral cutting block having defined therein (i) a pair of chamfer cutting slots, (ii) an anterior cutting slot spaced apart anteriorly from the pair of chamfer cutting slots, and (iii) a posterior cut surface spaced apart posteriorly from the pair of chamfer cutting slots,
a medial paddle spaced apart posteriorly from the posterior cut surface, the medial paddle having an alignment surface that extends parallel to the posterior cut surface of the 4-in-1 femoral cutting block in the superior/inferior direction and is configured to be positioned in contact with a medial posterior condyle of the patient's femur, and
a lateral paddle spaced apart posteriorly from the posterior cut surface, the lateral paddle having an alignment surface that extends parallel to the posterior cut surface of the 4-in-1 femoral cutting block in the superior/inferior direction and is configured to be positioned in contact with a lateral posterior condyle of the patient's femur.

2. The orthopaedic surgical instrument of claim 1, further comprising a plurality of shims, wherein:
each of the plurality of shims has a thickness that is different from the thickness of at least some of the other of the plurality of shims, and
each of the plurality of shims includes a locking mechanism that mates with a locking mechanism of the medial paddle and the lateral paddle such that each of the plurality of shims is configured to be separately coupled to both the medial paddle and the lateral paddle.

3. The orthopaedic surgical instrument of claim 1, further comprising a plurality of shims, wherein:
each of the plurality of shims has a thickness that is different from the thickness of at least some of the other of the plurality of shims, and
each of the plurality of shims includes a locking mechanism that mates with a locking mechanism of one or both of the medial paddle and the lateral paddle such that each of the plurality of shims is configured to be separately coupled to one or both of the medial paddle and the lateral paddle.

4. The orthopaedic surgical instrument of claim 1, further comprising a posterior saw capture surface, wherein:
the posterior saw capture surface is spaced apart posteriorly from the posterior cut surface, and
the posterior saw capture surface is spaced apart anteriorly from both the medial paddle and the lateral paddle.

5. The orthopaedic surgical instrument of claim 4, further comprising an alignment guide, wherein:
the alignment guide is selectively securable to, and removable from, the 4-in-1 femoral cutting block, and
each of the medial paddle, the lateral paddle, and the saw capture surface are formed in the alignment guide.

6. The orthopaedic surgical instrument of claim 5, wherein the alignment guide includes a connector configured to secure the alignment guide to the 4-in-1 femoral cutting block.

7. The orthopaedic surgical instrument of claim 1, wherein:
the medial paddle has a longitudinal axis extending in the superior/inferior direction, and
the lateral paddle has a longitudinal axis extending in the superior/inferior direction so as to be parallel to the longitudinal axis of the medial paddle.

8. An orthopaedic surgical instrument for use in the surgical preparation of a patient's femur during performance of an orthopaedic knee procedure, the orthopaedic surgical instrument comprising:
a 4-in-1 femoral cutting block having defined therein (i) a pair of chamfer cutting slots, (ii) an anterior cutting slot spaced apart anteriorly from the pair of chamfer cutting slots, and (iii) a posterior cut surface spaced apart posteriorly from the pair of chamfer cutting slots, and
alignment guide removably secured to a posterior end of the 4-in-1 femoral cutting block, the alignment guide comprising: (i) a medial paddle spaced apart posteriorly from the posterior cut surface, the medial paddle having an alignment surface that extends parallel to the posterior cut surface of the 4-in-1 femoral cutting block in the superior/inferior direction and is configured to be positioned in contact with a medial posterior condyle of the patient's femur, and (ii) a lateral paddle spaced apart posteriorly from the posterior cut surface, the lateral paddle having an alignment surface that extends parallel to the posterior cut surface of the 4-in-1 femoral cutting block in the superior/inferior direction and is configured to be positioned in contact with a lateral posterior condyle of the patient's femur.

9. The orthopaedic surgical instrument of claim 8, further comprising a plurality of shims, wherein:
each of the plurality of shims has a thickness that is different from the thickness of at least some of the other of the plurality of shims, and
each of the plurality of shims includes a locking mechanism that mates with a locking mechanism of the medial paddle and the lateral paddle such that each of the plurality of shims is configured to be separately coupled to both the medial paddle and the lateral paddle.

10. The orthopaedic surgical instrument of claim 8, further comprising a plurality of shims, wherein:
each of the plurality of shims has a thickness that is different from the thickness of at least some of the other of the plurality of shims, and
each of the plurality of shims includes a locking mechanism that mates with a locking mechanism of one or both of the medial paddle and the lateral paddle such that each of the plurality of shims is configured to be separately coupled to one or both of the medial paddle and the lateral paddle.

11. The orthopaedic surgical instrument of claim 8, wherein:
the alignment guide further includes a posterior saw capture surface,
the posterior saw capture surface is spaced apart posteriorly from the posterior cut surface of the 4-in-1 femoral cutting block, and
the posterior saw capture surface is spaced apart anteriorly from both the medial paddle and the lateral paddle.

12. The orthopaedic surgical instrument of claim 8, wherein the alignment guide further includes a connector configured to secure the alignment guide to the 4-in-1 femoral cutting block.

13. The orthopaedic surgical instrument of claim 8, wherein:
the medial paddle has a longitudinal axis extending in the superior/inferior direction, and
the lateral paddle has a longitudinal axis extending in the superior/inferior direction so as to be parallel to the longitudinal axis of the medial paddle.

14. A method of surgically preparing a patient's femur during performance of an orthopaedic knee procedure, the method comprising:
resecting a distal end of the patient's femur so as to create a resected planar surface,
determining an amount of cartilage loss on a posterior femoral condyle of the patient's femur,
selecting a shim having a thickness that corresponds to the determined amount of cartilage loss on the posterior femoral condyle of the patient's femur from a plurality of shims of differing thicknesses,
installing the selected shim onto a paddle positioned on a posterior end of a 4-in-1 femoral cutting block, and
positioning the 4-in-1 femoral cutting block on the resected distal end of the patient's femur such that (i) a backside surface of the 4-in-1 femoral cutting block contacts the resected planar surface, and (ii) the shim installed on the paddle contacts the posterior femoral condyle of the patient's femur.

15. The method of claim 14, further comprising:
pinning the 4-in-1 femoral cutting block to the patient's femur subsequent to positioning thereof on the resected distal end thereof, and
performing further resections on the patient's femur by use of the pinned 4-in-1 femoral cutting block.

16. The method of claim 15, wherein performing further resections comprises performing an anterior resection, a distal resection, and a pair of chamfer resections on the patient's femur by use of the pinned 4-in-1 femoral cutting block.

17. The method of claim 14, wherein:
determining the amount of cartilage loss comprises determining an amount of cartilage loss on a medial posterior femoral condyle of the patient's femur,
selecting the shim comprises selecting a shim having a thickness that corresponds to the determined amount of cartilage loss on the medial posterior femoral condyle of the patient's femur from a plurality of shims of differing thicknesses,
installing the selected shim comprises installing the selected shim onto a medial paddle positioned on the posterior end of the 4-in-1 femoral cutting block, and
positioning the 4-in-1 femoral cutting block comprises positioning the 4-in-1 femoral cutting block on the resected distal end of the patient's femur such that (i) the backside surface of the 4-in-1 femoral cutting block contacts the resected planar surface, and (ii) the shim installed on the medial paddle contacts the medial posterior femoral condyle of the patient's femur.

18. The method of claim 14, wherein:
determining the amount of cartilage loss comprises determining an amount of cartilage loss on a lateral posterior femoral condyle of the patient's femur,
selecting the shim comprises selecting a shim having a thickness that corresponds to the determined amount of cartilage loss on the lateral posterior femoral condyle of the patient's femur from a plurality of shims of differing thicknesses,
installing the selected shim comprises installing the selected shim onto a lateral paddle positioned on the posterior end of the 4-in-1 femoral cutting block, and
positioning the 4-in-1 femoral cutting block comprises positioning the 4-in-1 femoral cutting block on the resected distal end of the patient's femur such that (i) the backside surface of the 4-in-1 femoral cutting block contacts the resected planar surface, and (ii) the shim installed on the lateral paddle contacts the lateral posterior femoral condyle of the patient's femur.

19. The method of claim 14, wherein:
determining the amount of cartilage loss comprises determining an amount of cartilage loss on both a medial posterior femoral condyle of the patient's femur and a lateral posterior femoral condyle of the patient's femur,
selecting the shim comprises (i) selecting a medial shim having a thickness that corresponds to the determined amount of cartilage loss on the medial posterior femoral condyle of the patient's femur from a plurality of shims of differing thicknesses, and (ii) selecting a lateral shim having a thickness that corresponds to the determined amount of cartilage loss on the lateral posterior femoral condyle of the patient's femur from the plurality of shims of differing thicknesses,
installing the selected shim comprises (i) installing the selected medial shim onto a medial paddle positioned on a posterior end of a 4-in-1 femoral cutting block, and (ii) installing the selected lateral shim onto a lateral paddle positioned on the posterior end of the 4-in-1 femoral cutting block, and
positioning the 4-in-1 femoral cutting block comprises positioning the 4-in-1 femoral cutting block on the resected distal end of the patient's femur such that (i) the backside surface of the 4-in-1 femoral cutting block contacts the resected planar surface, (ii) the medial shim installed on the medial paddle contacts the medial posterior femoral condyle of the patient's femur, and (iii) the lateral shim installed on the lateral paddle contacts the lateral posterior femoral condyle of the patient's femur.

20. The method of claim 14, wherein determining the amount of cartilage loss on the posterior femoral condyle of the patient's femur comprises advancing a depth probe into cartilage of the posterior femoral condyle of the patient's femur.

21. A method of surgically preparing a patient's femur during performance of an orthopaedic knee procedure, the method comprising:
determining an amount of cartilage loss on a posterior femoral condyle of the patient's femur,
selecting a shim having a thickness that corresponds to the determined amount of cartilage loss on the posterior femoral condyle of the patient's femur from a plurality of shims of differing thicknesses,
installing the selected shim onto a paddle positioned on a posterior end of a femoral cutting block, and
positioning the femoral cutting block on a distal end of the patient's femur such that (i) a backside surface of the femoral cutting block contacts the distal end of the patient's femur, and (ii) the shim installed on the paddle contacts the posterior femoral condyle of the patient's femur.

22. The method of claim 21, further comprising:
pinning the femoral cutting block to the patient's femur subsequent to positioning the femoral cutting block on the distal end thereof, and
performing resections on the patient's femur by use of the pinned femoral cutting block.

23. The method of claim 21, wherein:
determining the amount of cartilage loss comprises determining an amount of cartilage loss on a medial posterior femoral condyle of the patient's femur,
selecting the shim comprises selecting a shim having a thickness that corresponds to the determined amount of cartilage loss on the medial posterior femoral condyle of the patient's femur from a plurality of shims of differing thicknesses,
installing the selected shim comprises installing the selected shim onto a medial paddle positioned on the posterior end of the femoral cutting block, and
positioning the femoral cutting block comprises positioning the femoral cutting block on the distal end of the patient's femur such that (i) the backside surface of the femoral cutting block contacts the distal end of the patient's femur, and (ii) the shim installed on the medial paddle contacts the medial posterior femoral condyle of the patient's femur.

24. The method of claim 21, wherein:
determining the amount of cartilage loss comprises determining an amount of cartilage loss on a lateral posterior femoral condyle of the patient's femur,
selecting the shim comprises selecting a shim having a thickness that corresponds to the determined amount of cartilage loss on the lateral posterior femoral condyle of the patient's femur from a plurality of shims of differing thicknesses,
installing the selected shim comprises installing the selected shim onto a lateral paddle positioned on the posterior end of the femoral cutting block, and
positioning the femoral cutting block comprises positioning the femoral cutting block on the distal end of the patient's femur such that (i) the backside surface of the femoral cutting block contacts the distal end of the patient's femur, and (ii) the shim installed on the lateral paddle contacts the lateral posterior femoral condyle of the patient's femur.

25. The method of claim 21, wherein determining the amount of cartilage loss on the posterior femoral condyle of the patient's femur comprises advancing a depth probe into cartilage of the posterior femoral condyle of the patient's femur.
